(19) United States
(12) Patent Application Publication
(10) Pub. No.: US 2014/0031598 A1
(43) Pub. Date: Jan. 30, 2014

(54) PROCESS FOR THE PRODUCTION OF AROMATIC HYDROCARBONS

(71) Applicants: Sujata Degaleesan, Katy, TX (US); Glenn Charles Komplin, Katy, TX (US); Vishwanath Subramaniam, Houston, TX (US); Kuochen Tsai, Katy, TX (US)

(72) Inventors: Sujata Degaleesan, Katy, TX (US); Glenn Charles Komplin, Katy, TX (US); Vishwanath Subramaniam, Houston, TX (US); Kuochen Tsai, Katy, TX (US)

(21) Appl. No.: 13/989,450

(22) PCT Filed: Nov. 21, 2011
(86) PCT No.: PCT/US2011/061577

§ 371 Date: Oct. 15, 2013

Related U.S. Application Data
(60) Provisional application No. 61/416,784, filed on Nov. 24, 2010.

(51) Int. Cl. C07C 2/88; C07C 17/013

(52) U.S. Cl. CPC C07C 2/88; C07C 17/013
USPC 570/252; 585/321

(57) ABSTRACT

A process comprising feeding bromine into a first reactor; feeding low molecular weight alkanes into the first reactor; and withdrawing alkyl bromides from the first reactor wherein the bromine and low molecular weight alkanes are fed through an apparatus that rapidly mixes the bromine and low molecular weight alkanes. A process is disclosed further comprising reacting the alkyl bromides to form aromatic hydrocarbons.

### FIELD OF THE INVENTION

This invention relates to a process for the production of aromatic hydrocarbons by bromination of low molecular weight alkanes, particularly methane. More particularly, the invention relates to a process that incorporates an apparatus to facilitate rapid mixing of the bromine and low molecular weight alkanes in the bromination step.

### BACKGROUND OF THE INVENTION

U.S. Pat. No. 7,244,867 describes a process for converting lower molecular weight alkanes, including methane, natural gas or ethane, propane, etc., into higher molecular weight hydrocarbons, including aromatics, by bromination to form alkyl bromides and hydrobromic acid which are then reacted over a crystalline alumino-silicate catalyst to form the higher molecular weight hydrocarbons and hydrobromic acid. Hydrobromic acid is recovered by contacting the reaction product stream with water and then converted to bromine for recycle. The higher molecular weight hydrocarbons are recovered.

The bromination to produce alkyl bromides produces monobrominated as well as polybrominated compounds. Monobrominated alkyl bromides are preferred in the next step of the reaction. The process typically has a reproportionation reactor to convert polybrominated alkyl bromides to monobrominated alkyl bromides.

It can be seen that it would be advantageous to provide a process that produced fewer polybrominated alkyl bromides, as this would improve the subsequent reaction steps and allow for a smaller reproportionation reactor. The present invention provides such a process.

### SUMMARY OF THE INVENTION

The present invention provides a process comprising feeding bromine into a first reactor; feeding low molecular weight alkanes into the first reactor; and withdrawing alkyl bromides from the first reactor wherein the bromine and low molecular weight alkanes are fed through an apparatus that rapidly mixes the bromine and low molecular weight alkanes.

### BRIEF DESCRIPTION OF THE DRAWING

FIG. 1 is a flow diagram illustrating the process of the present invention.

FIG. 2 is a diagram of an embodiment of the rapid mixing apparatus. FIG. 2a provides a view of the apparatus and FIG. 2b provides a view of the cross section of the apparatus.

### DETAILED DESCRIPTION OF THE INVENTION

The present invention provides a process for the production of aromatic compounds from low molecular weight alkanes, primarily methane. Other alkanes, such as ethane, propane, butane, and pentane, may be mixed in with the methane or may be fed as the primary low molecular weight alkane. First, at least one low molecular weight alkane, preferably methane, is halogenated by reacting it with a halogen, preferably bromine. The monohaloalkane, preferably monobromomethane, which is produced thereby may be contacted with a suitable coupling catalyst which causes the monohaloalkane to react with itself to produce higher molecular weight hydrocarbons such as aromatics. A small amount of methane may also be produced. The aromatic compounds, such as benzene, toluene and xylenes, may be separated from the methane. Higher molecular weight aromatic hydrocarbons may also be produced in the coupling step, such as those containing nine or more carbon atoms. These C₉₊ hydrocarbons may be processed as described below and converted into olefins and/or more desirable aromatic hydrocarbons such as benzene, toluene and/or xylenes.

The hydrocarbon feed may be comprised of a low molecular weight alkane. Low molecular weight alkanes include methane, ethane and propane, as well as butane and pentane. The preferred feed is natural gas which is comprised of methane and often contains smaller amounts of ethane, propane and other hydrocarbons. In one embodiment, the preferred feed is methane. In a second embodiment, the preferred feed is propane.

Higher molecular weight hydrocarbons are defined herein as those hydrocarbons having a greater number of carbon atoms than the components of the lower molecular weight hydrocarbon feedstock. Higher molecular weight hydrocarbons include aromatic hydrocarbons, especially benzene, toluene and xylenes (hereinafter referred to as “BTX”).

In a preferred embodiment, the coupling reaction may be carried out such that the production of aromatic hydrocarbons, specifically BTX, is maximized. The production of aromatic hydrocarbons may be achieved by the use of a suitable coupling catalyst under suitable operating conditions.

Representative halogens include bromine and chlorine. It is also contemplated that fluorine and iodine may be used but not necessarily with equivalent results. Some of the problems associated with fluorine possibly may be addressed by using dilute streams of fluorine. It is expected that more vigorous reaction conditions will be required for alkyl fluorides to couple and form higher molecular weight hydrocarbons. Similarly, problems associated with iodine (such as the endothermic nature of some iodine reactions) may likely be addressed by carrying out the halogenation and/or coupling reactions at higher temperatures and/or pressures. The use of bromine or chlorine is preferred and the use of bromine is most preferred. While the following description may only refer to bromine, bromination and/or bromomethanes, the description is applicable to the use of other halogens and halomethanes as well.

Bromination of the methane (methane will be used in the following description but other alkanes may be used or may be present as discussed above) may be carried out in an open pipe, a fixed bed reactor, a tube-and-shell reactor or another suitable reactor, preferably at a temperature and pressure where the bromination products and reactants are gases. Rapid mixing between bromine and methane is preferred to help prevent over-bromination and coking. For example, the reaction pressure may be from about 100 to about 5000 kPa and the temperature may be from about 150 to about 600° C., more preferably from about 350 to about 550° C. and even more preferably from about 400 to about 515° C. Higher temperatures tend to favor coke formation and lower temperatures require larger reactors. Methane bromination may be initiated using heat or light with thermal means being preferred.

Rapid mixing of the bromine and methane as these streams enter the bromination reactor is important to limiting the production of polybrominated alkyl bromides, for example, di- and tri-bromomethane. Maldistribution of the bromine will typically result in the production of polybrominated species.

One embodiment of a rapid mixing apparatus for mixing the bromine and methane on entry into the bromination reactor is depicted in FIG. 2. The apparatus 200 comprises two concentric tubes; an inner tube 210 and an outer tube 220. The longitudinal axes of the tubes are preferably aligned. The outer tube has an inlet end 230 where methane is introduced and an outlet end 240 where a mixture of methane, bromine, hydrobromic acid and methyl bromides are discharged. The inner tube has an inlet end 250 where bromine is introduced into the tube. The inner tube also has a plurality of openings 212 to allow the bromine to contact the methane in the outer tube, rapidly mix and react.

The openings are preferably circular, but can be of any shape or design known to one of ordinary skill in the art. The openings may be spaced as desired. There are preferably at least 3 openings located in a plane perpendicular to the longitudinal axis of the inner tube. Openings may be located along more than one plane perpendicular to the longitudinal axis of the inner tube. For example, there may be 4 openings located in a plane perpendicular to the longitudinal axis and another 4 openings in a different plane perpendicular to the longitudinal axis.

The openings are typically from 0.5 to 3 mm in diameter, preferably from 1 to 2 mm in diameter. It is understood that the size of the openings may be outside this range depending on the size of the apparatus and the flow of bromine and methane.

The inner tube preferably has a tapered tip at the end opposite the inlet end to improve the flow and mixing dynamics in the apparatus.

A halogenation catalyst may also be used in the halogenations step. In an embodiment, the reactor may contain a halogenation catalyst such as a zeolite, amorphous alumino-silicate, acidic zirconia, tungstenates, solid phosphoric acids, metal oxides, mixed metal oxides, metal halides, mixed metal halides (the metal in such cases being for example nickel, copper, cerium, cobalt, etc.) and/or other catalysts as described in U.S. Pat. Nos. 3,935,289 and 4,971,664, each of which is herein incorporated by reference in its entirety. Specific catalysts include a metal bromide (for example, sodium bromide, potassium bromide, copper bromide, nickel bromide, magnesium bromide and calcium bromide), a metal oxide (for example, silicon dioxide, zirconium dioxide and aluminum trioxide) or metal (for example, platinum, palladium, ruthenium, iridium, or rhodium) to help generate the desired brominated methane.

The bromination reaction product comprises monobromomethane, HBr and also small amounts of dibromomethane and tribromomethane. If desired, the HBr may be removed prior to coupling. The presence of large concentrations of the polybrominated species in the feed to the coupling reactor may decrease bromine efficiency and result in an undesirable increase in coke formation. In many applications, such as the production of aromatics and light olefins, it is desirable to feed only monobromomethane to the coupling reactor to improve the conversion to the final higher molecular weight hydrocarbon products. In an embodiment of the invention, a separation step is added after the halogenation reactor in which the monobromomethane is separated from the other bromomethanes. The di- and tribromomethane species may be recycled to the bromination reactor. One separation method is described in U.S. Published Patent Application No. 2007/02388909, which is herein incorporated by reference in its entirety. Preferably, the separation is carried out by distillation. The di- and tribromomethanes are higher boiling than the monobromomethane, unreacted methane and HBr, which is also made by the bromination reaction:

CH₄+Br₂→CH₃Br+HBr

In a preferred embodiment, the polybromomethanes may be recycled to the halogenation reaction and preferably reproportionated to convert them to monobromomethane. The polybromomethanes contain two or more bromine atoms per molecule. Reproportionation may be accomplished according to U.S. Published Patent Application 2007/0238909 which is herein incorporated by reference in its entirety. Reactive reproportionation is accomplished by allowing the methane feedstock and any recycled alkanes to react with the polybrominated methane species from the halogenation reactor, preferably in the substantial absence of molecular halogen. Reproportionation may be carried out in a separate reactor or in a region of the halogenation reactor.

The bromination and coupling reactions may be carried out in separate reactors or the process may be carried out in an integrated reactor, for example, in a zone reactor as described in U.S. Pat. No. 6,525,230 which is herein incorporated by reference in its entirety. In this case, halogenation of methane may occur within one zone of the reactor and may be followed by a coupling step in which the liberated hydrobromic acid may be adsorbed within the material that catalyzes condensation of the halogenated hydrocarbon. Hydrocarbon coupling may take place within this zone of the reactor and may yield the product higher molecular weight hydrocarbons including aromatic hydrocarbons. It is preferred that separate reactors be used for bromination and coupling because operating conditions may be optimized for the individual steps and this allows for the possibility of removing polybrominated-methane before the coupling step.

Coupling of monobromomethane may be carried out in a fixed bed, fluidized bed or other suitable reactor. The temperature may range from about 150 to about 600° C., preferably from about 300 to about 550° C., most preferably from about 350 to about 475° C., and the pressure may range from about 10 to about 3500 kPa absolute, preferably about 100 to about 2500 kPa absolute. In general, a relatively long residence time favors conversion of reactants to products as well as product selectivity to BTX, while a short residence time means higher throughput and possibly improved economics. It is possible to change product selectivity by changing the catalyst, altering the reaction temperature, pressure and/or altering the residence time in the reactor. Low molecular weight alkanes may also exit the coupling reactor. These low molecular weight alkanes may be comprised of ethane and propane but may also include methane and a small amount of C₄₋₅ alkanes and smaller amounts of alkenes. Some of these may be recycled to the bromination reactor but preferably the low molecular weight alkanes may be directed to the cracking step.

Preferred coupling catalysts for use in the present invention are described in U.S. Patent Application No. 2007/0238909 and U.S. Pat. No. 7,244,867, each of which is herein incorporated by reference in its entirety.

A metal-oxygen cataloreactant may also be used to facilitate the coupling reaction.

The term “metal-oxygen cataloreactant” is used herein to mean a cataloreactant material containing both metal and oxygen. Such cataloreactants are described in detail in U.S. Published Patent Application Nos. 2005/0038310 and 2005/0171393 which are herein incorporated by reference in their entirety. Examples of metal-oxygen cataloreactants given therein include zeolites, doped zeolites, metal oxides, metal oxide-impregnated zeolites and mixtures thereof. Nonlimiting examples of dopants include alkaline earth metals, such as calcium, magnesium, and barium and their oxides and/or hydroxides and metals such as manganese, iron, cobalt, nickel, molybdenum, lanthanum, and lead, and their oxides.

Hydrogen bromide may also be produced along with monobromomethane in the bromination reactor. The hydrogen bromide may be carried over to the coupling reactor or, if desired, may be separated before coupling. The products of the coupling reaction may include higher molecular weight hydrocarbons, especially BTX and C₂₊ alkanes and likely some alkenes, C₉₊ aromatics and hydrogen bromide. In a preferred embodiment, the hydrogen bromide may be separated from the higher molecular weight hydrocarbon products by distillation.

The coupling reaction product, higher molecular hydrocarbons and hydrogen bromide may be sent to an absorption column wherein the hydrogen bromide may be absorbed in water using a packed column or other contacting device. Input water in the product stream may be contacted either in co-current or countercurrent flow with countercurrent flow preferred for its improved efficiency. One method for removing the hydrogen bromide from the higher molecular weight hydrocarbon reaction product is described in U.S. Pat. No. 7,244,867 which is herein incorporated by reference in its entirety. Hydrogen bromide present in the C₂₊ alkanes and alkenes stream or the product stream from the bromination reactor may also be removed therefrom by this method.

In an embodiment, the hydrogen bromide is recovered by displacement as a gas from its aqueous solution in the presence of an electrolyte that shares a common ion or an ion that has a higher hydration energy than hydrogen bromide. Also aqueous solutions of metal bromides such as calcium bromide, magnesium bromide, sodium bromide, potassium bromide, etc. may be used as extractive agents.

In another embodiment, catalytic halogen generation is carried out by reacting hydrogen bromide and molecular oxygen over a suitable catalyst. The oxygen source may be air, pure oxygen or enriched air. A number of materials have been identified as halogen generation catalysts. It is possible to use oxides, halides, and/or oxyhalides of one or more metals, such as magnesium, calcium, barium, chromium, manganese, iron, cobalt, nickel, copper, zinc, palladium, platinum, etc. After the HBr is separated from the hydrocarbon products, it may be reacted to produce bromine for recycle to the bromination step. Catalysts and methods for regeneration of the bromine are described in detail in U.S. Published Application 2007/0238909 which is herein incorporated by reference in its entirety. Recovery of bromine is also described therein.

In addition to the higher molecular weight hydrocarbons and the hydrogen bromide, other materials may exit from the coupling reactor. These include methane, light ends (C₂₊ alkanes and alkenes) and heavy ends (aromatic C₉₊ hydrocarbons and a small amount of nonaromatic C₆₊ hydrocarbons, usually less than 1%). The methane may be separated from these other materials (e.g., by distillation) and recycled to the bromination reactor. The C₂₊ alkanes, and optionally the alkenes, may be separated from the other materials and introduced into an alkane cracker which produces ethylene and/or propylene and possibly other olefins such as butenes, pentenes, etc. The C₂₊ alkanes and alkenes stream may contain some HBr which may be removed prior to cracking. The C₉₊ aromatic hydrocarbons may be hydrogenated. The hydrogen for hydrogenation may be that produced in the alkane cracker. The resulting hydrogenated C₉₊ stream may be cracked in a conventional cracker to produce additional olefins and/or aromatic hydrocarbons. Alternatively, the C₉₊ aromatic hydrocarbons may be converted to xylenes by reproportionation with toluene, hydrodealkylated to BTX or they may be upgraded by a combination of these two steps.

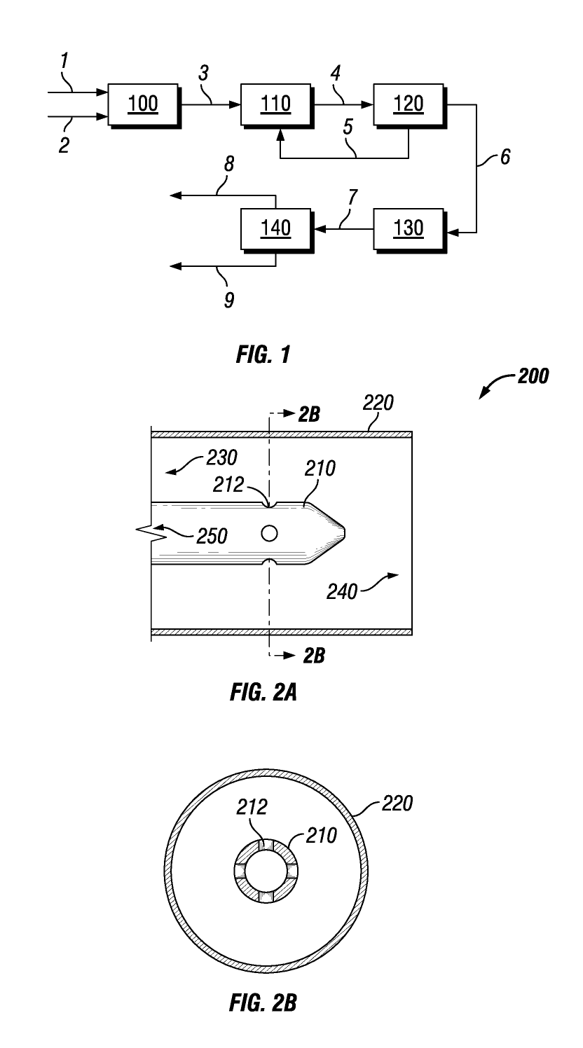

One embodiment of the invention is illustrated in FIG. 1. Methane is delivered through line 1 to the bromination reactor 100 at 30 barg (3000 kPag) and ambient temperature. This methane stream may be combined with a methane recycle stream. The methane stream is heated to 450° C., and fed to the bromination reactor 100. Bromine liquid is pumped from storage in line 2, vaporized and heated to 250° C., and fed into the bromination reactor 100. A rapid mixing apparatus is preferably used to mix the methane and bromine as they enter the bromination reactor.

In the bromination reactor 100, bromine reacts adiabatically with methane to form methyl bromide, methyl dibromide, methyl tribromide, and hydrogen bromide. In this example, the reactor does not utilize a catalyst. During normal operation, a small amount of coke is produced. The bromination reactor 100 is comprised of at least 2 parallel reactor trains to allow for one train to be decoked while the other train(s) remains in normal operation.

A gas mixture containing methyl bromides, hydrogen bromide and unreacted methane, exits the bromination reactor 100 through line 3 at 510° C. and 30 barg (3000 kPag) and enters the reproportionation reactor 110. The reproportionation product gas stream 4 is cooled and fractionated in a conventional distillation column 120 to separate polybrominated hydrocarbons from the other reproportionation products. Polybrominated hydrocarbons, recovered from distillation column 120, are fed to the reproportionation reactor 110 through line 5 where di- and tri-substituted methyl bromide and other polybrominated hydrocarbons react adiabatically with unreacted methane to form monobromomethane. In this example, the reproportionation reactor 110 does not utilize a catalyst.

The remaining components of the reproportionation product stream 4 (primarily monobromomethane, hydrogen bromide, and unreacted methane) are recovered as a separate stream 6, vaporized, reheated to 400° C., and fed to the coupling reactor 130.

In the coupling reactor 130, monobromomethane reacts adiabatically over a catalyst, preferably manganese-based, at a temperature of 425° C. and 25 barg (2500 kPag) to produce a mixture of compounds comprised predominately of benzene, toluene, xylenes, ethane, propane, butane, and pentanes. The coupling reactor 130 may be comprised of multiple fixed bed catalytic reactors operating on a reaction/regeneration cycle. During the reaction phase, monobromomethane reacts to form mixed products. At the same time, coke is formed and gradually deactivates the catalyst.

Product gas from the coupling reactor 130 is directed through line 7 and cooled and fractionated in conventional distillation column 140 to produce two streams. The higher boiling stream, 9, is comprised primarily of benzene, toluene, and xylenes. The lower boiling stream, 8, is comprised primarily of methane, ethane, propane, butanes, pentanes, and hydrogen bromide.

Additional purification, treatment and recycle steps may be carried out, but these are not described here.

1. A process comprising
a. feeding bromine into a first reactor
b. feeding low molecular weight alkanes into the first reactor; and
c. withdrawing alkyl bromides from the first reactor
wherein the bromine and low molecular weight alkanes are fed through an apparatus that rapidly mixes the bromine and low molecular weight alkanes.

2. A process as claimed in claim 1 wherein the bromine is introduced into the reactor in a gaseous phase.

3. A process as claimed in claim 1 wherein the bromine is introduced into the reactor in a liquid phase.

4. A process as claimed in claim 1 wherein the bromine and low molecular weight alkanes are fed together through the same apparatus.

5. A process as claimed in claim 1 wherein the apparatus comprises two concentric pipes; one central pipe having an inner and outer wall and a second pipe having a larger diameter than the first pipe and having an inner and an outer wall, and the bromine is passed through the central pipe and the low molecular weight alkanes are passed through the region between the outer wall of the central pipe and the inner wall of the second pipe.

6. A process as claimed in claim 5 wherein the apparatus further comprises a tapered tip such that at the outlet of the outer pipe, the diameter of the outer pipe is less than the average diameter of the outer pipe along its entire length.

7. A process as claimed in claim 1 wherein the bromine and low molecular weight alkanes are fed into the first reactor at a molar ratio of bromine to low molecular weight alkanes of from 0.5:1 to 3:1.

8. A process as claimed in claim 1 wherein the first reactor does not contain catalyst.

9. A process as claimed in claim 1 wherein the low molecular weight alkanes comprise at least 50 mole percent propane.

10. A process as claimed in claim 1 wherein the low molecular weight alkanes comprise at least 80 mole percent propane.

11. A process as claimed in claim 1 wherein the alkyl bromides comprise at least 50 mole percent monobrominated alkanes.

12. A process as claimed in claim 1 wherein the alkyl bromides comprise at most 20 mole percent polybrominated alkanes.

13. A process as claimed in claim 5 wherein the second pipe has a plurality of injection points that provide fluid communication between the region inside the inner wall of the central pipe and the region between the outer wall of the central pipe and the inner wall of the second pipe.

14. A process as claimed in claim 13 wherein the injection points are located in at least one plane perpendicular to the longitudinal axis of the central pipe.

15. A process as claimed in claim 14 wherein the injection points are located in at least two planes perpendicular to the longitudinal axis of the central pipe.

16. A process as claimed in claim 1 further comprising reacting the alkyl bromides with hydrobromic acid over a catalyst to produce higher molecular weight hydrocarbons.
